(19) United States
(12) Patent Application Publication
(10) Pub. No.: US 2024/0369573 A1
(43) Pub. Date: Nov. 7, 2024

(54) METHODS FOR EMBRYO SCREENING

(71) Applicant: Shahryar K. KAVOUSSI, Austin, TX (US)

(72) Inventor: Shahryar K. KAVOUSSI, Austin, TX (US)

(21) Appl. No.: 18/683,325

(22) PCT Filed: Aug. 12, 2022
(86) PCT No.: PCT/US2022/074935

§ 371 Date: Feb. 13, 2024

Related U.S. Application Data
(60) Provisional application No. 63/318,919, filed on Mar. 11, 2022.
(60) Provisional application No. 63/233,086, filed on Aug. 13, 2021.

(51) Int. Cl. G01N 33/68; G01N 33/543

(52) U.S. Cl. CPC G01N 33/689; G01N 33/54306; G01N 2800/36; G01N 2800/387

(57) ABSTRACT

Methods of assessing obstetric outcomes of embryos based on the expression of alpha-fetoprotein (AFP), e.g., as measured in a biopsy from an embryo at the blastocyst stage, blastocoel fluid, or embryo culture conditioned media, such as blastocoel fluid conditioned media (BFCM), are provided.

This application claims the benefit of U.S. Provisional Patent Application No. 63/233,086, filed Aug. 13, 2021, and U.S. Provisional Patent Application No. 63/318,919, filed Mar. 11, 2022, the entirety of which are incorporated herein by reference.

### BACKGROUND OF THE INVENTION

### 1. Field of the Invention

The present invention relates generally to the field of molecular biology and medicine. More particularly, it concerns methods of screening embryos for viability and improved obstetric outcomes.

### 2. Description of Related Art

Alpha-fetoprotein (AFP) is found in both fetal serum and also amniotic fluid. This protein is produced early in gestation by the fetal yolk sac and then later in the liver and gastrointestinal tract. The exact biological function of AFP is presently unknown. During a baby's development, some AFP passes through the placenta and into the mother's blood. An AFP test measures the level of AFP in pregnant women during the second trimester of pregnancy. Too much or too little AFP in a mother's blood may be sign of a birth defect or other condition.

The Alpha-Fetoprotein Test (AFT) test is a screening test that examines the level of alpha-fetoprotein in the mother's blood during pregnancy. AFT is performed by measuring the level of AFP by a fetus can be detected in maternal blood during the second trimester. More specifically, the AFP test is normally be performed between the 14th and 22nd weeks of pregnancy; however, it may be more accurate during the 16th to 18th week.

The AFT tests for high or low levels of alpha-fetoprotein. The results are combined with the mother's age and ethnicity in order to assess the probabilities of potential genetic disorders. High levels of AFP may suggest the developing baby has a neural tube defect such as spina bifida or anencephaly. High levels of AFP may also suggest a defect with the esophagus or failure of the fetal abdomen to close. However, the most common reason for elevated AFP levels is inaccurate dating of the pregnancy. Low levels of AFP and abnormal levels of hCG and estriol may indicate the developing baby has Trisomy 21 (Down syndrome), Trisomy 18 (Edwards Syndrome) or another type of chromosome abnormality.

The AFT is currently only used as a screening test and is not considered, by itself, a diagnostic test. If an AFT shows a high or low level of maternal serum AFP, then additional diagnostic tests are normally performed. These additional diagnostic tests may include an additional AFT, an ultrasound test, or an amniocentesis (a genetic screen of a small amount of amniotic fluid obtained during the second or third trimester).

While AFT is clinically useful, this test includes several significant limitations. For example, the test is not performed until the second trimester, which is considered by many to be very late to identify significant problems for an unborn child. Clearly, there is a need for improved methods for testing for levels of fetal AFP.

### SUMMARY OF THE INVENTION

The present disclosure, in some aspects, overcomes limitations in the prior at by providing improved methods for measuring expression or expression levels of AFP in an unborn fetus or embryo. In some aspects, it has been observed that AFP can be detected in blastocoel fluid (BF) and blastocoel fluid conditioned media (BFCM). It is anticipated that high or low expression of AFP in BF or BFCM can indicate an increased risk of a physiological problem with the fetus or an adverse obstetric outcome (e.g., resulting in loss of the embryo during a pregnancy). For example, levels of AFP in the BF or BFCM either above or below a control level may indicate increased risk of spina bifida, anencephaly, failure of the fetal abdomen to close, increased risk of a chromosomal abnormality, aneuploidy, Trisomy 21 (Down syndrome), Trisomy 18 (Edwards Syndrome), or other aneuploidies. In some aspects, methods of selecting embryos for use in an in vitro fertilization (IVF) procedure are provided. In some aspects, the methods provided herein can be used for preimplantation prenatal screening (PPS).

An aspect of the present disclosure relates to an in vitro method of screening an embryo, comprising: (i) obtaining in vitro an embryo at the blastocyst stage of development, and (ii) measuring the expression of alpha-fetoprotein (AFP) in one or more embryo cells, in the blastocoel fluid, and/or in embryo culture fluid that has been used to store or culture the embryo; wherein an increased or decreased expression of AFP above a first control level or below a second control level indicates an increased risk of a genetic disease in the embryo or an adverse obstetric outcome if the embryo is implanted into a mammalian subject. In some embodiments, the expression of AFP is measured in the blastocoel fluid. In some embodiments, the expression of AFP is measured in the embryo culture fluid or blastocoel fluid conditioned media (BFCM). The expression of AFP may be measured in one or more embryo cells. The one or more embryo cells may comprise or consist of one or more trophoblast or trophectoderm cells. The one or more trophoblast cells may comprise or consist of syncytiotrophoblast cells. The one or more embryo cells may comprise or consist of one or more inner cell mass cells. In some embodiments, the adverse obstetric outcome is abnormal placentation, spontaneous miscarriage, small for gestational age (SGA), intrauterine growth restriction (IUGR), intrauterine fetal demise (IUFD), or pre-eclampsia. In some embodiments, increased expression of AFP above the first control level indicates increased risk of spina bifida, anencephaly, or failure of fetal abdomen closure. In some embodiments, decreased expression of AFP below the second control level indicates increased risk of a chromosomal abnormality (e.g., aneuploidy, Trisomy 21 (Down syndrome), or Trisomy 18 (Edwards Syndrome)). In some embodiments, the genetic disease is spina bifida, anencephaly, failure of fetal abdomen closure, an aneuploidy, Trisomy 21 (Down syndrome), or Trisomy 18 (Edwards Syndrome). The measuring may be performed via detecting or measuring mRNA that encodes AFP. In some embodiments, the measuring is performed via Northern blot analysis, nuclease protection assays (NPA), in situ hybridization, reverse transcription-polymerase chain reaction (RT-PCR), semi-quantitative PCR, or next-generation mRNA sequencing (mRNA-Seq). In some embodiments, the reverse transcription-polymerase chain reaction (RT-PCR) is further defined as a reverse transcription quantitative PCR (RT-qPCR). In some embodiments, the measuring is performed via detecting or measuring AFP protein is a Western blot, high-performance liquid chromatography (HPLC), liquid chromatography-mass spectrometry (LC/MS), enzyme-linked immunosorbent assay (ELISA), protein immunostaining, or electrochemiluminescence immunoassay (ECLIA). In some embodiments, the measuring is performed via ELISA. The method may further comprise testing the embryo for aneuploidies (PGT-A). In some embodiments, the method further comprises selecting an embryo for implantation into the mammalian subject. In some embodiments, the embryo is implanted into the mammalian subject as part of an in vitro fertilization (IVF) method. The embryo may have been generated from an oocyte obtained from a donor. In some embodiments, the donor is the mammalian subject. In some embodiments, the donor is a first human subject, and wherein the mammalian subject is a second human subject. The mammalian subject may be a cow, sheep, horse, dog, cat, lion, panther, ferret, goat, or pig. The mammalian subject may be a domesticated animal. In some embodiments, the mammalian subject is an endangered species. In some embodiments, the mammalian subject is a human. The method may further comprise performing an additional screening method or an additional genetic test of the embryo. The additional genetic testing may comprise an amniocentesis. In some embodiments, the additional screening method comprises measuring the expression of pregnancy-associated plasma protein-A (PAPPA), a placental growth factor (PlGF), and/or one or more members of the a disintegrin or metalloproteinase (ADAM) family in one or more embryo cells of the embryo, in the blastocoel fluid, and/or in embryo culture fluid that has been used to store or culture the embryo. ADAMs (a disintegrin and metalloproteinases), originally also known as MDC proteins (metalloproteinase/disintegrin/cysteine-rich), belong to the Metzincins superfamily of metalloproteases (e.g., Giebeler et al. (2016)). The additional genetic testing may comprise or consist of testing for the presence of a genetic disease in the embryo. In some embodiments, the embryo is further defined as a human embryo, and wherein the genetic disease is Huntington's disease, sickle cell anemia, muscular dystrophy, cystic fibrosis (CF), a BRCA1 mutation, a BRCA2 mutation, fragile-X syndrome, or Tay-Sachs disease.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

The present disclosure, in some aspects, provides improved methods for selecting embryos for implantation (e.g., for an in vitro fertilization (IVF) procedure) that have improved viability or a reduced risk of an adverse obstetric outcome. In some aspects, increased or decreased expression of AFP in an embryonic tissue, the blastocoel fluid, or embryo culture media as measured by either mRNA or protein levels can indicate an increased risk of an adverse obstetric outcome (e.g., abnormal placentation, increased risk of spontaneous miscarriage, small for gestational age (SGA), intrauterine growth restriction (IUGR), intrauterine fetal demise (IUFD), or pre-eclampsia). Increased levels of AFP in the BF or BFCM above or below a control level may indicate increased risk of spina bifida, anencephaly, failure of the fetal abdomen to close, a chromosomal abnormality, aneuploidy, Trisomy 21 (Down syndrome), Trisomy 18 (Edwards Syndrome), or other aneuploidies.

### II. ALPHA-FETOPROTEIN (AFP)

AFP (also referred to as AFPD, FETA, or HPalpha fetoprotein; Homo sapiens Gene ID: 174) is a glycoprotein molecule that has some similarity to albumin. AFP is produced by the fetal liver, fetal liver and yolk sac cells, and to a smaller extent by fetal gastrointestinal tract and kidney. Its biological role is unclear and factors that may influence its concentrations in neonates are only partially identified. AFP expression can be used as a diagnostic marker, especially in certain tumors, such as hepatocellular carcinoma, liver cancers, nonseminomatous germ cell tumors, and yolk sac tumors. The synthesis of AFP is almost completely absent in normal adult tissues but is reactivated in cancers, particularly in primary hepatoma and in yolk sac tumors.

AFP expression in adults has been associated with a variety of cancers. For example, increased serum concentration of alpha-fetoprotein (AFP) can be found in benign and malignant liver diseases, in yolk sac tumors, and in several nonhepatic neoplasms at advanced stage. The frequency and level of elevated serum AFP are highest in hepatocellular carcinoma (HCC) and yolk sac tumors (e.g., Wu, 1990).

### II. AFP DETECTION METHODS

A variety of techniques can be used to detect mRNA encoding AFP or AFP protein. The mRNA encoding AFP or AFP protein may be measured in more embryo cells (e.g., trophoblast or trophectoderm cells, syncytiotrophoblast cells, or inner cell mass cells) in the blastocoel fluid, and/or in embryo culture fluid that has been used to store or culture the embryo.

A variety of methods may be used to detect or measure mRNA that encodes AFP. For example, in various embodiments, the method may be Northern blot analysis, nuclease protection assays (NPA), in situ hybridization, reverse transcription-polymerase chain reaction (RT-PCR), semi-quantitative PCR, or next-generation mRNA sequencing (mRNA-Seq).

In some embodiments, RT-PCR is performed to measure AFP mRNA. For example, in some embodiments, the following method may be used. RNA can be extracted with TRIZOL (guanidinium thiocyanate) from a sample. The samples can be centrifuged for 10 min at 11,200 g and the resulting RNA pellet was washed once with 70% ethanol and resuspended in 40 μl of diethyl pyrocarbonate-treated water. cDNA can be synthesized, e.g., using a Takara cDNA synthesis kit (Takara Bio, Inc., Otsu, Japan) following the manufacturer's protocol. qPCR can be conducted using SYBR. In some embodiments, the following primers can be used for amplification of AFP: forward primer GCAGAGGAGATGTGCTGGATTG (SEQ ID NO:1) and reverse primer CGTGGTCAGTTTGCAGCATTCTG (SEQ ID NO:2), e.g., at about 56° C. In some embodiments, the reverse transcription-polymerase chain reaction (RT-PCR) is a reverse transcription quantitative PCR (RT-qPCR) methodology or a semi-quantitative PCR methodology. Semi-quantitative PCR methods are known in the art and include those described, e.g., in Chen et al., 1999.

In some embodiments, next-generation sequencing is used to measure AFP mRNA via RNA-seq (RNA-sequencing). RNA-seq methods using next-generation sequencing can be used to quantify expression of genes (e.g., Mortazavi et al., 2008; Trapnell et al., 2010). Next-generation sequencing methods that may be used include: sequencing methods that identifying DNA bases based on the emission of a unique fluorescent signal as nucleic acids are added to a nucleic acid chain (e.g., by llumina (Solexa)), pyrosequencing methods (e.g., 454 sequencing), and detection of nucleic acid incorporation by detection of hydrogen ions with a semiconductor (e.g., Ion Torrent methods). Next generation sequencing includes massively parallel signature sequencing, polony sequencing, cPAS sequencing, SOLiD sequencing, DNA nanoball sequencing, and SMRT PacBio single molecule real time sequencing (e.g., by Pacific Bioscience).

Additional methods that can be used to measure AFP mRNA include quantitative real-time RT-PCR. Real-time RT-PCR has been successfully use in a wide variety of fields for some time. This method can be used for measuring mRNA levels of in vivo low copy number targets of interest. Benefits of this procedure over other methods for measuring RNA include its sensitivity, large dynamic range, the potential for high throughout as well as accurate quantification (Huggett, et al., 2005).

In some embodiments, AFP protein levels are measured from in more embryo cells (e.g., trophoblast or trophectoderm cells, syncytiotrophoblast cells, or inner cell mass cells) in the blastocoel fluid, and/or in embryo culture fluid that has been used to store or culture the embryo. A variety of methods for detection of AFP protein may be used. For example, methods that may be used include: Western blot, high-performance liquid chromatography (HPLC), liquid chromatography-mass spectrometry (LC/MS), enzyme-linked immunosorbent assay (ELISA), protein immunostaining, or electrochemiluminescence immunoassay “ECLIA” (Elecsys). In some embodiments, microfluidics may be used to quantify the AFP protein.

In some aspects, the level of expression of AFP is compared to a control level or normal level of expression. In some embodiments, the control level of normal level of expression can be established in experiments and quantified at the embryonic level, for example with using culture medium as a negative control. Increased or decreased expression of AFP can be indicative of an adverse obstetric outcome (e.g., is abnormal placentation, spontaneous miscarriage, small for gestational age (SGA), intrauterine growth restriction (IUGR), intrauterine fetal demise (IUFD), or pre-eclampsia). Expression levels of AFP in the BF or BFCM (e.g., as measured based on mRNA or protein levels) that are either increased above a first control level or decreased below a second control level may indicate increased risk of spina bifida, anencephaly, failure of the fetal abdomen to close, a chromosomal abnormality, aneuploidy, Trisomy 21 (Down syndrome), Trisomy 18 (Edwards Syndrome), or other aneuploidies. Thus, the methods provided herein can be used in selecting an embryo for implantation to increase the chances of a healthy, live birth as part of an IVF protocol.

### III. EMBRYONIC TISSUES FOR AFP TESTING

It is anticipated that a variety of embryonic tissues at the blastocyst stage of development, the blastocoel fluid, and/or the embryo culture media, can be used to measure AFP expression in a method as disclosed here, e.g., to identify embryos for use in an IVF procedure. In some embodiments, a tissue sample or biopsy is obtained the embryo is at day 3, 4, 5, 6, of 7 of development; for example, a biopsy of one cell from day 3 may be obtained, or a biopsy of one or multiple cells (e.g., 1, 2, 3, 4 cells) from a day 5, day 6, or day 7 embryo may be obtained. The embryonic tissue may comprise or consist of trophoblasts, such as syncytiotrophoblast cells.

Embryonic tissues at the blastocyst stage of development include trophoblasts, the blastocyst cavity (blastocoele), and inner cell mass (embryoblast). Trophoblasts (trophectoderm cells) form the outer layer of the blastocyst and are normally observed within four to six days after fertilization in humans. Some trophectoderm cells can differentiate into become extraembryonic structures, and trophectoderm cells do not directly contribute to the embryo. Syncytiotrophoblast cells are a type of trophoblast cell that form the epithelial covering of vascular embryonic placental villi, which invade the wall of the uterus and are involved in obtaining nutrients from the mother.

In some embodiments, one or more cells from the inner cell mass can be used as the tissue biopsy for measuring AFP levels. In some instances, the embryonic tissue biopsy is not the inner cell mass, since these cells will form the developing mammalian subject; nonetheless, a recent study supports the idea that inner cell mass cells from a blastocyst may be obtained without adversely impairing the developing embryo (Scott et al., 2013).

In some embodiments, a tissue biopsy is obtained from the embryo to detect and measure expression of AFP. For example, in order to access embryonic cells for biopsy, which may be trophectoderm (TE) and/or inner cell mass (ICM) cells, laser pulses can be applied to the zona pellucidae which surround a blastocyst stage embryo and then the laser is applied to the junctions between embryonic cells in order to obtain a biopsy. The biopsied embryonic cells can then be removed (e.g., by pipette), and can be placed into a buffer solution. AFP can then be measured as described herein, e.g., by detecting or quantifying AFP mRNA or protein. After obtaining the blastocyst biopsy, the embryo may collapse, resulting in blastocoel fluid being extruded out into the surrounding medium.

Embryo culture medium containing blastocoel fluid (ECB), also referred to as blastocoel fluid conditioned medium (BFCM) can also be obtained and used to test for AFP expression. A variety of methods can be used to obtain a sample of blastocoel fluid. Methods for obtaining ECB are described, e.g., in Li et al., 2018 or Stigliani, 2014. In some embodiments, ECB can be obtained via the following method. An infrared laser can be used to lase a small breech in the zone pellucida (ZP) far away from the inner cell mass to release the blastocoels fluid into the culture medium. The released blastocoel fluid mixed with culture media (e.g., about 25 μl) can be transferred to RNase-DNase-free tubes for subsequent analysis (e.g., PCR). To prevent media contamination, different Pasteur pipettes can be used for each sample.

Trophectoderm cells can be obtained by a variety of techniques known in the art. For example, a laser or biopsy pipette can be used to obtain the trophectoderm cells. In some embodiments, trophectoderm cells were encouraged to herniate from the zona by applying gentle suction with the biopsy pipette. One or multiple trophectoderm cells (e.g., 1, 2, 3, 4, or 5 cells) may be dissected from a blastocyst using a laser (e.g., four laser pulses of 3 seconds in duration). The biopsied cells can be placed immediately in RNase-DNase-free tubes for further analysis of AFP expression as described herein (e.g., using PCR etc.).

In some embodiments, embryo culture medium containing blastocoel fluid (ECB) is used. Blastocoel fluid conditioned media, which is routinely discarded, can be collected and saved (e.g., post biopsy) from a Day 5, Day 6, or Day 7 blastocyst stage mammalian embryo (such as a human embryo). In some embodiments, the biopsy is obtained from embryos that are being analyzed for possible implantation in a patient undergoing IVF, for example along with a preimplantation genetic testing for aneuploidy. Additional methods for obtaining blastocoel fluid conditioned media that may be used include, e.g., Chosed et al., 2019; Vera-Rodriguez et al., 2018; Rule et al., 2018; and Xu et al., 2016.

### IV. IN VITRO FERTILIZATION (IVF)

Measuring expression of AFP in an embryo at the blastocyst stage to predict an obstetric outcome may be performed as part of an IVF procedure. In vitro fertilization includes a variety of techniques for assisting with the conception and birth of a child. In some preferred embodiments, the IVF procedure is for a human patient. Nonetheless, techniques disclosed herein can be applied to a wide variety of mammals, including domesticated animals, including cows, horses, dogs, cats, sheep, goats, or pigs, or endangered species, such as various lions and panthers.

Generally, during IVF mature eggs are collected from ovaries from a mammalian and fertilized by sperm in a lab. The fertilized egg (embryo) or eggs (embryos) are then transferred to a uterus. One full cycle of IVF takes about three weeks. These steps can be separated into different parts, if desired. The IVF procedure may involve intracytoplasmic sperm injection (ICSI) (e.g., Neti et al., 2014). IVF has been used in various forms since its introduction in 1978.

A variety of additional tests may be performed on the embryo prior to implantation in an IVF procedure. For example, blastocoel fluid or a tissue biopsy from the embryo (e.g., trophectoderm cells that are also used to assess AFP expression as described herein) can be analyzed with fluorescence in situ hybridization (FISH), array comparative genomic hybridization (aCGH), single-nucleotide polymorphism (SNP) arrays, multiplex quantitative PCR or next generation sequencing (NGS) to test for aneuploidy, determine the chromosomal status of the embryo, and/or to facilitate selection of desired embryos for implantation.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—The Expression of Alpha-Fetoprotein in Human Blastocoel Fluid-Conditioned Media In Vitro

The aim of this study was to determine if alpha-fetoprotein (AFP), a protein known to be produced in the fetal liver and yolk sac detected in maternal serum as well as in amniotic fluid to assess for fetal neural tube defects (NTD) and aneuploidy, is expressed in blastocoel fluid-conditioned media (BFCM) at the embryonic blastocyst stage.

Materials and Methods: BFCM was obtained from blastocyst stage embryos following standard, routine controlled ovarian stimulation and subsequent IVF embryology processes. Good quality blastocysts (n=40) had undergone trophectoderm biopsy for preimplantation genetic testing for aneuploidy (PGT-A) prior to blastocyst vitrification and BFCM collection. BFCM samples (n=40) were assessed for human AFP protein expression using an AFP Human ELISA Kit. Fisher's Exact Test was used for statistical analysis.

Results: AFP was detected in 12.5% of the BFCM samples (5/40), with a range of 1.69 pg/mL to 20.5 pg/mL. Of the blastocysts which had AFP detected in BFCM, 80% (4/5) had PGT-A results indicating aneuploidy; of the blastocysts with no AFP detected in BFCM, 57% (20/35) had PGT-A results indicating aneuploidy; there was no significant difference in aneuploidy status between groups (p=0.63). Results are provided below in Table 1.

TABLE 1
AFP in BFCM Samples

| BFCM | AFP | Day | Embryo | PGT |
|---|---|---|---|---|
| Samples | (pg/mL) | 5 or 6 | Grade | result |
| 1 | 1.692308 | 5 | 3AB | Abnormal |
| 2 | 8.307692 | 5 | 2AA | Abnormal |
| 3 | 10.46154 | 5 | 2AA | Abnormal |
| 4 | 0 | 5 | 3BB | Abnormal |
| 5 | 0 | 5 | 4AA | Normal |
| 6 | 0 | 5 | 3AA | Normal |
| 7 | 0 | 5 | 3AA | Abnormal |
| 8 | 0 | 5 | 3AB | Abnormal |
| 9 | 0 | 5 | 3AB | Normal |
| 10 | 0 | 5 | 3BB | Normal |
| 11 | 0 | 5 | 2AA | Normal |
| 12 | 0 | 5 | 2AB | Normal |
| 13 | 0 | 5 | 2BB | Abnormal |
| 14 | 0 | 6 | 3AB | Abnormal |
| 15 | 0 | 5 | 4AB | Normal |
| 16 | 0 | 6 | 2AB | Normal |
| 17 | 0 | 6 | 3AA | Abnormal |
| 18 | 0 | 6 | 3BB | Abnormal |
| 19 | 0 | 5 | 3AA | Normal |
| 20 | 0 | 5 | 3AA | Normal |
| 21 | 0 | 5 | 3AA | Normal |
| 22 | 20.53846 | 5 | 3AA | Normal |
| 23 | 0 | 5 | 2AA | Abnormal |
| 24 | 0 | 6 | 2AB | Abnormal |
| 25 | 0 | 5 | 3AA | Abnormal |
| 26 | 0 | 5 | 3AA | Abnormal |
| 27 | 0 | 5 | 3AA | Abnormal |
| 28 | 0 | 5 | 3AA | Abnormal |
| 29 | 0 | 5 | 2AA | Abnormal |
| 30 | 0 | 5 | 2AB | Normal |
| 31 | 0 | 6 | 2BB | Normal |
| 32 | 0 | 6 | 3AA | Abnormal |
| 33 | 0 | 6 | 2AA | Abnormal |
| 34 | 0 | 6 | 3AB | Abnormal |
| 35 | 0 | 6 | 3AB | Normal |
| 36 | 0 | 6 | 2BB | Abnormal |
| 37 | 0 | 5 | 2AA | Abnormal |
| 38 | 1.923078 | 5 | 4AA | Abnormal |
| 39 | 0 | 5 | 3AA | Abnormal |
| 40 | 0 | 5 | 3AA | Normal |

Conclusions: These studies demonstrate the expression of AFP protein in BFCM. To our knowledge, this is the first study to report detection of AFP in BFCM from blastocyst stage embryos. Future, larger studies, particularly when more extended embryonic culture is available in clinical IVF, can be used to measure and statistically analyze expression of AFP at the embryonic stage. It is anticipated that increased expression can improve embryo transfer outcomes.

It is anticipated that AFP detection in BFCM can be used as a minimally-invasive way to assess for an embryo with minimal risk of developing NTD as a desired adjunct to embryonic euploidy in selecting the optimal embryo for uterine transfer.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Chen et al., Brain Research Protocols: 4(2), p. 132-139, 1999.
Chosed et al., Integr Mol Med, vol: 6, p. 1-4, 2019.
Giebeler et al. Toxins (Basel). 8(4):122, 2016.
Huggett, et al. Genes Immun.; 6(4):279-84, 2005.
Li et al. Scientific Reports, volume 8, Article number: 9275, 2018.
Mortazavi et al, Nat Methods, 5: 621-628, 2008.
Neti et al., Cell Calcium, 55(1): 24-37, 2014.
Rule et al., J Assist Reprod Genet.; 35(8):1497-1501, 2018.
Scott et al., Fertility & Sterility 100, 624, 2013.
Stigliani et al., Molecular Human Reproduction 20, 1238-1246, 2014.
Trapnell et al., Nat Biotechnol.; 28: 511-515, 2010.
Vera-Rodriguez et al., Human Reproduction, Vol. 33, No. 4 pp. 745-756, 2018.
Wu, Ann Clin Lab Sci Mar. 1, 1990 vol. 20 no. 2 98-105.
Xu et al., PNAS | vol. 113 | no. 42 | 11907-11912, 2016.

1. An in vitro method of screening an embryo, comprising:
(i) obtaining in vitro an embryo at the blastocyst stage of development, and
(ii) measuring the expression of alpha-fetoprotein (AFP) in one or more embryo cells, in the blastocoel fluid, and/or in embryo culture fluid that has been used to store or culture the embryo;
wherein an increased or decreased expression of AFP above a first control level or below a second control level indicates an increased risk of a genetic disease in the embryo or an adverse obstetric outcome if the embryo is implanted into a mammalian subject.

2. The method of claim 1, wherein the expression of AFP is measured in the blastocoel fluid.

3. The method of claim 1, wherein the expression of AFP is measured in the embryo culture fluid or blastocoel fluid conditioned media (BFCM).

4. The method of claim 1, wherein the expression of AFP is measured in one or more embryo cells.

5. The method of claim 4, wherein the one or more embryo cells comprise or consist of one or more trophoblast or trophectoderm cells.

6. The method of claim 5, wherein the one or more trophoblast cells comprise or consist of syncytiotrophoblast cells.

7. The method of claim 4, wherein the one or more embryo cells comprise or consist of one or more inner cell mass cells.

8. The method of claim 1, wherein the adverse obstetric outcome is abnormal placentation, spontaneous miscarriage, small for gestational age (SGA), intrauterine growth restriction (IUGR), intrauterine fetal demise (IUFD), or pre-eclampsia.

9. The method of claim 1, wherein increased expression of AFP above the first control level indicates increased risk of spina bifida, anencephaly, or failure of fetal abdomen closure.

10. The method of claim 1, wherein decreased expression of AFP below the second control level indicates increased risk of a chromosomal abnormality.

11. The method of claim 10, wherein the chromosomal abnormality is an aneuploidy, Trisomy 21 (Down syndrome), or Trisomy 18 (Edwards Syndrome).

12. The method of claim 1, wherein the genetic disease is spina bifida, anencephaly, failure of fetal abdomen closure, an aneuploidy, Trisomy 21 (Down syndrome), or Trisomy 18 (Edwards Syndrome).

13. The method of claim 1, wherein, the measuring is performed via detecting or measuring mRNA that encodes AFP.

14. The method of claim 13, wherein the measuring is performed via Northern blot analysis, nuclease protection assays (NPA), in situ hybridization, reverse transcription-polymerase chain reaction (RT-PCR), semi-quantitative PCR, or next-generation mRNA sequencing (mRNA-Seq).

15. The method of claim 14, wherein the reverse transcription-polymerase chain reaction (RT-PCR) is further defined as a reverse transcription quantitative PCR (RT-qPCR).

16. The method of claim 1, wherein, the measuring is performed via detecting or measuring AFP protein is a Western blot, high-performance liquid chromatography (HPLC), liquid chromatography-mass spectrometry (LC/MS), enzyme-linked immunosorbent assay (ELISA), protein immunostaining, or electrochemiluminescence immunoassay (ECLIA).

17. The method of claim 16, wherein the measuring is performed via ELISA.

18. The method of claim 1, wherein the method further comprises testing the embryo for aneuploidies (PGT-A).

19. The method of claim 1, wherein the method further comprises selecting an embryo for implantation into the mammalian subject.

20. The method of claim 1, wherein the embryo is implanted into the mammalian subject as part of an in vitro fertilization (IVF) method.

21. The method of claim 20, wherein the embryo has been generated from an oocyte obtained from a donor.

22. The method of claim 21, wherein the donor is the mammalian subject.

23. The method of claim 21, wherein the donor is a first human subject, and wherein the mammalian subject is a second human subject.

24. The method of claim 1, wherein the mammalian subject is a cow, sheep, horse, dog, cat, lion, panther, ferret, goat, or pig.

25-26. (canceled)

27. The method of claim 1, wherein the mammalian subject is a human.

28. The method of claim 1, wherein the method further comprises performing an additional screening method or an additional genetic test of the embryo.

29. The method of claim 28, wherein the additional genetic testing comprises an amniocentesis.

30. The method of claim 28, wherein the additional screening method comprises measuring the expression of pregnancy-associated plasma protein-A (PAPPA), a placental growth factor (PlGF), and/or one or more members of the a disintegrin and metalloproteinase (ADAM) family in one or more embryo cells of the embryo, in the blastocoel fluid, and/or in embryo culture fluid that has been used to store or culture the embryo.

31. The method of claim 28, wherein the additional genetic testing comprises or consists of testing for the presence of a genetic disease in the embryo.

32. The method of claim 31, wherein the embryo is further defined as a human embryo, and wherein the genetic disease is Huntington's disease, sickle cell anemia, muscular dystrophy, cystic fibrosis (CF), a BRCA1 mutation, a BRCA2 mutation, fragile-X syndrome, or Tay-Sachs disease.


SEQUENCE LISTING (2 sequences)

SEQ ID NO: 1 - DNA, 22 bp, synthetic construct
  source 1..22: mol_type = other DNA
gcagaggagatgtgctggattg

SEQ ID NO: 2 - DNA, 23 bp, synthetic construct
  source 1..23: mol_type = other DNA
cgtggtcagtttgcagcattctg